(19) United States
(12) Patent Application Publication
(10) Pub. No.: US 2021/0025889 A1
(43) Pub. Date: Jan. 28, 2021

(54) ELISA KIT FOR CLOSTRIDIUM NOVYI TYPE B

(71) Applicants: SHANDONG GENGMU BIOLOGICAL TECHNOLOGY CO., LTD., Shandong (CN); SHANDONG AGRICULTURAL UNIVERSITY, Shandong (CN); SHANDONG DELTA BIOENGINEERING CO., LTD., Shandong (CN)

(72) Inventors: Tongjie CHAI, Shandong (CN); Dezhi MENG, Shandong (CN); Liangmeng WEI, Shandong (CN)

(21) Appl. No.: 16/934,888

(22) Filed: Jul. 21, 2020

(30) Foreign Application Priority Data
Jul. 24, 2019 (CN) 201910670248.1

(51) Int. Cl. G01N 33/569

(52) U.S. Cl. CPC G01N 33/56911; G01N 2333/33; G01N 2469/20

(57) ABSTRACT

The present invention discloses an enzyme-linked immunosorbent assay (ELISA) kit for Clostridium novyi type B that adopts a suspension of Clostridium novyi type B as a coating antigen. The suspension of Clostridium novyi type B is prepared by the following method: Clostridium novyi type B is inoculated into a medium for enrichment cultivation; the resulting Clostridium novyi type B culture is centrifuged to collect bacteria; and the bacteria are washed and resuspended with carbonate buffer to obtain a suspension of Clostridium novyi type B. The kit of the present invention, which adopts intact Clostridium novyi bacteria as a coating antigen, can detect whether there are anti-Clostridium novyi antibodies in a sheep serum, with high sensitivity and specificity, and thus determine whether the sheep develops black disease. The present invention has detection results of strong specificity and excellent repeatability.

### CROSS REFERENCE TO RELATED APPLICATIONS

This application claims priority under 35 U.S.C. § 119 to Chinese Patent Application No. 201910670248.1, which was filed on Jul. 24, 2019, the entire contents of which is hereby expressly incorporated by reference in its entirety.

### TECHNICAL FIELD

The present invention relates to the technical field of detection of Clostridium novyi, and in particular, to an enzyme-linked immunosorbent assay (ELISA) kit for sheep Clostridium novyi type B.

### BACKGROUND

Black disease, also known as “infectious necrotic hepatitis”, is an acute, highly fatal infectious disease caused by Clostridium novyi type B. Clostridium novyi can cause gas gangrene in humans, malignant edema in animals, big head disease in sheep, and black disease in sheep, goats, cattle, horses and the like, with acute onset and high mortality. Therefore, Clostridium novyi is one of the most harmful biological factors for zoonotic diseases and imposes severe hazard on animal husbandry, thereby causing huge economic losses to the development of animal husbandry worldwide. The analysis and detection of black disease has not received much attention all the time, while in the clinical and scientific research processes, it is necessary to establish a rapid detection method for black disease. At present, there is no rapid detection method for black disease, which is not conducive to the development of scientific research and healthy animal husbandry.

Currently, the etiological diagnosis methods for black disease mainly include the PCR of pathogens, clinical necropsy to observe the pathological changes, or the like, but these methods are basically not suitable for the in-vivo diagnosis of affected sheep. There must be anti-Clostridium novyi antibodies in the serum of affected sheep, therefore, the detection of this antibody level by ELISA can effectively reflect the infection of sheep.

At present, there are few ELISA methods for detecting antibodies against black disease. In a common ELISA method for detecting antibodies, the coating antigen used includes prokaryotically-expressed protein and ultrasonic bacterial protein. The prokaryotically-expressed protein, when used as a coating antigen, will cross-react with another epidemic disease-positive serum due to its poor specificity. The ultrasonic bacterial lysate, when used as a coating antigen, cannot ensure the stability among batches, because there is significant difference among results obtained from different batches of bacterial lysate. Moreover, internal proteins released from the bacteria are prone to cause false-positive detection results.

### SUMMARY

In view of the above-mentioned shortcomings in the prior art, the present invention is intended to provide an ELISA kit for Clostridium novyi type B. The kit of the present invention, which adopts intact Clostridium novyi bacteria as a coating antigen, can detect whether there are anti-Clostridium novyi antibodies in a sheep serum, with high sensitivity and specificity, and thus determine whether the sheep develops black disease. The present invention has detection results of strong specificity and excellent repeatability, and thus can help quickly make a diagnosis and take emergency therapeutic measures after the onset of sheep.

To achieve the above objective, the present invention adopts the following technical solutions.

In a first aspect of the present invention, an ELISA kit for Clostridium novyi type B is provided, and the ELISA kit uses a suspension of Clostridium novyi type B as a coating antigen.

Preferably, the suspension of Clostridium novyi type B is prepared by the following method:

Clostridium novyi type B is inoculated into an enrichment medium for cultivation; the resulting Clostridium novyi type B culture is centrifuged to collect bacteria; and the bacteria are washed and resuspended with carbonate buffer to obtain a suspension of Clostridium novyi type B.

More preferably, the enrichment medium is prepared by the following method:

10 g of tryptone, 10 g of yeast extract powder, 5 g of potassium phosphate, 10 g of glucose and 12 g of dried meat particles are dissolved in 1,000 mL of distilled water, and the resulting solution is sterilized at 120° C. for 15 min to obtain a basic medium;

1 g of vitamin K1 is dissolved in 99 ml of absolute ethanol, and the resulting solution is sterilized by filtration to obtain a vitamin K1 solution;

0.5 g of heme is dissolved in 1 mL of 1 mol/L sodium hydroxide solution, then distilled water is added to achieve a final volume of 100 mL, the resulting solution is autoclaved at 121° C. to obtain a heme solution, and the heme solution is stored at 4° C. for use; and 5 mL of vitamin K1 solution and 10 mL of heme solution are aseptically added to the basic medium cooled to room temperature, and the pH is adjusted to 7.8 to 8.4 to obtain an enrichment medium.

More preferably, Clostridium novyi type B is cultivated at 37° C. for 3 days in an anaerobic environment with a gas composition of 80% nitrogen, 10% hydrogen and 10% carbon dioxide (all in volume percentage).

Moreover, the ELISA kit further includes: an ELISA plate, a blocking solution, a Clostridium novyi type B-positive serum, a Clostridium novyi type B-negative serum, an enzyme-labeled secondary antibody, a chromogenic substrate solution and a stop solution.

Preferably, the blocking solution is 5% skimmed milk powder.

Preferably, the enzyme-labeled secondary antibody is HRP-labeled goat anti-rabbit antibody (for positive serum) and HRP-labeled rabbit anti-goat antibody (for sheep serum to be tested).

In a second aspect of the present invention, a method for detecting anti-Clostridium novyi type B antibodies using the above ELISA kit is provided, and the method includes the following steps:

(1) diluting the suspension of Clostridium novyi type B 40 times with an antigen-coating buffer, and then coating the ELISA plate overnight at 4° C., where the antigen-coating buffer is prepared by the following method:

1.465 g of NaHCO₃ and 0.795 g of Na₂CO₃ are dissolved in 400 mL of deionized water, the resulting solution is diluted to 500 mL, and the pH is adjusted to 9.5 with NaOH;

(2) washing the ELISA plate with PBST solution, and then performing the blocking with the blocking solution at 37° C. for 2 h;

(3) adding diluted Clostridium novyi type B-positive serum, Clostridium novyi type B-negative serum and to-be-tested serum separately, and incubating the plate at 37° C. for 1 h;

(4) adding an enzyme-labeled secondary antibody diluted at 1:10,000, and incubating the plate at 37° C. for 1 h;

(5) adding a chromogenic substrate solution, and conducting reaction at 37° C. for 15 min in the dark; and

(6) adding a stop solution to stop the reaction, reading the absorbance at 450 nm, and determining the test result.

Preferably, in step (3), Clostridium novyi type B-positive serum, Clostridium novyi type B-negative serum and to-be-tested serum are all diluted at 1:100.

Preferably, in step (6), the test result is determined according to the following criterion: if OD value of the to-be-tested sample is greater than 0.531, it is determined as positive; and if OD value of the to-be-tested sample is less than 0.531, it is determined as negative.

It should be noted that, in addition to the rapid diagnosis of black disease, the above method can also be used for the development of drugs and vaccines for treating diseases caused by Clostridium novyi type B.

The present invention has the following beneficial effects:

The ELISA kit for Clostridium novyi type B in the present invention has advantages, such as high sensitivity, strong specificity, excellent stability and simple operations, and can be used for detecting anti-Clostridium novyi type B antibodies, thereby achieving the rapid and effective detection of black disease.

### DETAILED DESCRIPTION

It should be noted that the following detailed description is exemplary and aims to further describe the present invention. Unless otherwise specified, all technical and scientific terms used herein have the same meaning as commonly understood by one of ordinary skill in the technical field to which the present application belongs.

As described in the background section, there are few studies on the rapid detection of black disease at home and abroad. Existing ELISA methods for detecting antibodies generally have disadvantages, such as low specificity, poor stability, and tendency to false-positive test results.

In view of this, the present invention is intended to provide an ELISA kit for Clostridium novyi type B, and to establish an ELISA method for rapidly detecting Clostridium novyi type B, with strong specificity, high sensitivity, excellent repeatability, low cost and simplicity, thereby offering technical support for the rapid diagnosis and the prevention and control of black disease.

Due to the difficulty for enrichment cultivation of Clostridium novyi, a desired Clostridium novyi recombinant protein is prepared by the prokaryotic expression of the Clostridium novyi recombinant protein using the reported sequence for Clostridium novyi and a modern biosynthesis technology. The preparation of a Clostridium novyi recombinant protein can break the current bottleneck of research on Clostridium novyi and accelerate the research on diseases caused by Clostridium novyi and related technologies. However, it is impossible to verify whether the Clostridium novyi recombinant protein has the same biological activity as the natural protein. Although the prokaryotic expression technology is mature, it is not uncommon to have cases where the expressed protein does not work. The prokaryotically-expressed protein, when used as a coating antigen, will cross-react with another epidemic disease-positive serum due to its poor specificity. The ultrasonic bacterial lysate, when used as a coating antigen, cannot ensure the stability among batches, because there is significant difference among results obtained from different batches of bacterial lysate. Moreover, internal proteins released from the bacteria are prone to cause false-positive detection results. Therefore, the present invention still subjects Clostridium novyi to enrichment cultivation and directly use the Clostridium novyi bacteria obtained from enrichment cultivation as a coating antigen to avoid these problems. Moreover, the recombinant protein, only targeting one type of bacterial protein, exotoxin protein or bacterial structure protein, cannot guarantee the reliability of the test results. At onset, bacteria are often first detected in sheep, and then exotoxins are produced from proliferation. Therefore, an ELISA detection method using bacteria as a coating antigen has higher accuracy.

However, the difficulty for directly using bacteria as a coating antigen lies in the proliferation of bacteria and the determination of the bacterial suspension concentration. The ELISA kit of the present invention uses Clostridium novyi type B bacteria as a coating antigen. The rapid and accurate detection of Clostridium novyi type B is achieved by determining the coating antigen concentration and the dilution of the to-be-tested serum, defining the critical value for positive serum and negative serum, and then establishing and assembling ELISA kit for Clostridium novyi type B.

In order to enable those skilled in the art to more clearly understand the technical solution of the present application, the technical solution of the present application is described in detail below with reference to a specific example.

The test materials used in the example of the present invention are all conventional test materials in the art, and can be commercially purchased.

The enrichment medium in the example of the present invention is prepared by the following method:

10 g of tryptone, 10 g of yeast extract powder, 5 g of potassium phosphate, 10 g of glucose and 12 g of dried meat particles are dissolved in 1,000 mL of distilled water, and the resulting solution is sterilized at 120° C. for 15 min to obtain a basic medium;

1 g of vitamin K1 is dissolved in 99 ml of absolute ethanol, and the resulting solution is sterilized by filtration to obtain a vitamin K1 solution;

0.5 g of heme is dissolved in 1 mL of 1 mol/L sodium hydroxide solution, then distilled water is added to achieve a final volume of 100 mL, the resulting solution is autoclaved at 121° C. to obtain a heme solution, and the heme solution is stored at 4° C. for use; and

5 mL of vitamin K1 solution and 10 mL of heme solution are aseptically added to the basic medium cooled to room temperature, and the pH is adjusted to 7.8 to 8.4 to obtain an enrichment medium.

### Example 1

1. Establishment of an ELISA Detection Method:

Step 1. Preparation of Coating Antigen

Clostridium novyi was inoculated into an enrichment medium made for Clostridium novyi in the laboratory, and subjected to enrichment cultivation at 37° C. for 3 days in an anaerobic environment with a gas composition of 80% nitrogen, 10% hydrogen and 10% carbon dioxide (all in volume percentage); the resulting culture was centrifuged at 10,000 r/min to 12,000 r/min for 3 min to 5 min to collect bacteria; and the bacteria were resuspended with carbonate buffer to obtain a bacterial suspension, namely, a coating antigen.

Step 2. Coating of the ELISA Plate

The bacterial suspension was added to the ELISA plate at 100 μL/well and placed overnight at 4° C. After the coating, PBST was added to the ELISA plate at 200 μL/well, and the plate was shaken gently for 3 min to 5 min. Then PBST was discarded, and the plate was repeatedly washed 3 times. The residual PBST in the well was removed by patting after the last wash.

Step 3. Blocking

5% skimmed milk powder was added to the ELISA plate at 100 μL/well for blocking at 37° C. for 2 h. After the blocking was completed, the plate was washed as in step 2.

Step 4. Incubation of the Primary Antibody in Serum

The to-be-tested serum was diluted at 1:100 and then added to the ELISA plate at 100 μL/well, and the plate was incubated at 37° C. for 1 h. After the incubation was completed, the plate was washed as in step 2.

Step 5. Incubation of the Enzyme-Labeled Secondary Antibody

The enzyme-labeled secondary antibody was diluted at 1:10,000 with PBST and then added to the ELISA plate at 100 μL/well, and the plate was incubated at 37° C. for 1 h. After the incubation was completed, the plate was washed as in step 2.

Step 6. Color Development

TMB chromogenic substrate solution, stored at 4° C. in the dark, was added to the ELISA plate at 100 μL/well, and the plate was incubated at 37° C. for 15 min.

Step 7. Stopping of the Reaction and Determination of the Results

2 mmol/L concentrated H₂SO₄, stored at room temperature, was added to the ELISA plate at 100 μL/well, and the plate was read on a microplate reader at 450 nm.

2. Determination of the Coating Concentration of Antigen

The cultivated and purified Clostridium novyi bacteria were used as the coating antigen, and the self-made positive serum was used as the positive control. The checkerboard titration was performed on a microplate reader. The Clostridium novyi bacteria were resuspended with carbonate buffer, and according to the serial dilution method, the ELISA plate was coated overnight at 4° C. with the Clostridium novyi bacteria at 100 μL/well in the longitudinal direction, with one column for each concentration. According to the above ELISA detection method, the optimal coating concentration for antigen was determined by the maximum P/N value. According to the test results, when the suspension of Clostridium novyi was diluted to 40 times, the corresponding bacterial concentration is the optimal coating concentration for antigen. The specific test results are shown in Table 1.

TABLE 1
Test results for the coating antigen concentration

| Bacterial | OD_{450 nm} values corresponding to different | | |
|---|---|---|---|
| concentration | coating concentrations for antigen | | |
| (serial dilution) | Negative serum | Positive serum | PZN value |
| 1:10 | 0.1700 | 1.2120 | 7.130 |
| 1:20 | 0.1629 | 1.1429 | 7.016 |
| 1:40 | 0.1538 | 1.0995 | 7.149 |
| 1:80 | 0.1485 | 0.9360 | 6.303 |
| 1:160 | 0.1382 | 0.8440 | 6.107 |

Due to the complex composition of the bacterial solution, it is difficult to remove substances such as dried meat particles, resulting in inaccurate OD values. Under the same cultivation conditions, the bacterial solutions have similar initial concentrations that can be regarded as the same. In the case where the bacteria cannot be cleaned, the present invention limits the concentration of a bacterial suspension according to the dilution ratio.

3. Determination of Critical Value

According to the ELISA operation steps in 1, 39 standard negative serum samples were diluted at 1:100 to obtain primary antibody serum samples, and OD_{450 nm} was determined for these samples. Average value (X̅) and standard deviation (SD) were calculated, and the critical value was the sum of X̅ and 3 SD. The critical value determined in the research was 0.531. The specific test results are shown in Table 2.

TABLE 2
Test results for critical value

| Sample | | Sample | | Sample | |
|---|---|---|---|---|---|
| No. | OD_{450 nm} | No. | OD_{450 nm} | No. | OD_{450 nm} |
| 1 | 0.2581 | 2 | 0.4612 | 3 | 0.2471 |
| 4 | 0.2707 | 5 | 0.4640 | 6 | 0.2727 |
| 7 | 0.2505 | 8 | 0.4772 | 9 | 0.2790 |
| 10 | 0.2635 | 11 | 0.4687 | 12 | 0.2577 |
| 13 | 0.2828 | 14 | 0.2861 | 15 | 0.3162 |
| 16 | 0.2574 | 17 | 0.2395 | 18 | 0.3352 |
| 19 | 0.3748 | 20 | 0.4323 | 21 | 0.2868 |
| 22 | 0.3460 | 23 | 0.3743 | 24 | 0.3248 |
| 25 | 0.3933 | 26 | 0.4117 | 27 | 0.3084 |
| 28 | 0.3315 | 29 | 0.3154 | 30 | 0.2864 |
| 31 | 0.2043 | 32 | 0.2118 | 33 | 0.2795 |
| 34 | 0.2944 | 35 | 0.2881 | 36 | 0.2817 |
| 37 | 0.2952 | 38 | 0.3014 | 39 | 0.3000 |

4. Specificity Assay

According to the ELISA operation steps in 1, serum samples of rabbits separately infected with Escherichia coli, Salmonella and Riemerella anatipestifer stored in the laboratory were detected. Serum was diluted at 1:100, and OD_{450 nm} was determined. Standard black disease-positive and -negative serum samples were adopted as positive and negative controls respectively. The detection results showed that Escherichia coli-, Salmonella- and Riemerella anatipestifer-positive serum samples all had an OD_{450 nm} value less than the positive critical value of 0.531, indicating that the test method established in this study has excellent specificity. The specific test results are shown in Table 3.

TABLE 3
Specificity assay results

| | | | Riemerella | | | |
|---|---|---|---|---|---|---|
| | Escherichia coli- | Salmonella- | anatipestifer- | Black disease- | Black disease- | |
| Serum type | positive serum | positive serum | positive serum | positive serum | negative serum | Blank control |
| OD_{450 nm} | 0.1713 | 0.1635 | 0.1914 | 2.9785 | 0.2861 | 0.1357 |

5. Repeatability Assay

(1) Within-batch repeatability assay: According to the ELISA operation steps in 1, OD_{450 nm} was determined for 5 black disease-positive serum samples and 5 black disease-negative serum samples on the same ELISA plate, with 3 replicates for each serum sample. The within-batch coefficient of variation (CV) was calculated as follows: within-batch CV=(average value of SD/OD_{450 nm})×100%. The results showed that the within-batch CV was less than 10%. The specific test results are shown in Table 4.

TABLE 4
Results for within-batch repeatability assay

| | Positive serum | | Negative serum | |
|---|---|---|---|---|
| Sample No. | X̅ + SD | CV/% | X̅ + SD | CV/% |
| 1 | 1.0402 + 0.0531 | 5.10 | 0.2828 + 0.0137 | 4.85 |
| 2 | 0.9721 + 0.0321 | 3.30 | 0.2395 + 0.0079 | 3.31 |
| 3 | 1.5952 + 0.1042 | 6.53 | 0.2861 + 0.0040 | 1.41 |
| 4 | 0.8904 + 0.0108 | 1.21 | 0.2471 + 0.0043 | 1.73 |
| 5 | 1.1519 + 0.0387 | 3.36 | 0.2577 + 0.0056 | 2.16 |

(2) Between-batch repeatability assay: According to ELISA operation steps in 1, OD_{450 nm} was determined for 5 standard pseudotuberculosis-positive serum samples and 5 standard pseudotuberculosis-negative serum samples on ELISA plates coated in different batches, with 3 replicates for each serum sample. The between-batch coefficient of variation (CV) was calculated as follows: between-batch CV=(SD/OD_{450 nm})×100%. The results showed that the between-batch CV was less than 10%. The specific test results are shown in Table 5.

TABLE 5
Results for between-batch repeatability assay

| | Positive serum | | Negative serum | |
|---|---|---|---|---|
| Sample No. | X̅ + SD | CV/% | X̅ + SD | CV/% |
| 1 | 1.2378 + 0.0309 | 2.50 | 0.2581 + 0.0089 | 3.13 |
| 2 | 1.1893 + 0.0212 | 1.78 | 0.2707 + 0.0035 | 1.45 |
| 3 | 2.1464 + 0.1569 | 7.31 | 0.2505 + 0.0023 | 0.79 |
| 4 | 1.0216 + 0.0553 | 5.41 | 0.2635 + 0.0063 | 2.54 |
| 5 | 1.3762 + 0.0475 | 3.45 | 0.2868 + 0.0122 | 4.75 |

6. Assembly of ELISA Kit

The ELISA kit for black disease specifically consists of the follows:

(1) a blank 96-well ELISA plate;

(2) a pure Clostridium novyi sample, stored at −20° C. and diluted 40 times with coating buffer before use;

(3) a Clostridium novyi-positive serum and a Clostridium novyi-negative serum;

(4) horseradish peroxidase-labeled rabbit anti-goat antibody, diluted at 1:10,000 with sample diluent before use; and

(5) a TMB chromogenic substrate solution, a stop solution, a blocking solution, a sample diluent and a washing solution.

### Example 2 Detection of Clinical Samples

15 clinical blood samples and 5 healthy sheep blood samples were collected from a sheep farm with infected sheep. Serum was separated for OD_{450 nm} determination. The detection results showed that the serum of infected sheep had an OD_{450 nm} value greater than the positive critical value, and the serum of healthy sheep had an OD_{450 nm} value less than the positive critical value, which was consistent with the actual incidence (with an accuracy rate of 100%), indicating that the detection method established in this study is reliable. The specific test results are shown in Table 6.

TABLE 6
Detection results for clinical samples

| Group | OD_{450 nm} | | | | |
|---|---|---|---|---|---|
| Serum of infected sheep | 1.2378 | 1.1893 | 2.1464 | 1.0216 | 1.3762 |
| | 1.1292 | 1.0724 | 2.1904 | 1.2838 | 1.7475 |
| | 1.7051 | 2.0692 | 1.2108 | 1.5805 | 1.2975 |
| Serum of healthy sheep | 0.2581 | 0.2707 | 0.2635 | 0.2861 | 0.2828 |

| | |
|---|---|
| Positive control | 3.7136 to 4 |
| Negative control | 0.2395 to 0.2861 |
| Blank control | 0.1357 to 0.17 |

The foregoing is merely illustrative of the preferred examples of the present invention and is not intended to limit the present invention, and various changes and modifications may be made to the present invention by those skilled in the art. Any modifications, equivalent substitutions, improvements or the like made within the spirit and scope of the present invention should be included within the protection scope of the present invention.

1. An enzyme-linked immunosorbent assay (ELISA) kit for Clostridium novyi type B, wherein the ELISA kit uses a suspension of Clostridium novyi type B as a coating antigen.

2. The ELISA kit according to claim 1, wherein the suspension of Clostridium novyi type B is prepared by the following method:
Clostridium novyi type B is inoculated into an enrichment medium for cultivation; the resulting Clostridium novyi type B culture is centrifuged to collect bacteria; and the bacteria are washed and resuspended with carbonate buffer to obtain a suspension of Clostridium novyi type B.

3. The ELISA kit according to claim 2, wherein the enrichment medium is prepared by the following method:
10 g of tryptone, 10 g of yeast extract powder, 5 g of potassium phosphate, 10 g of glucose and 12 g of dried meat particles are dissolved in 1,000 mL of distilled water, and the resulting solution is sterilized at 120° C. for 15 min to obtain a basic medium;
1 g of vitamin K1 is dissolved in 99 ml of absolute ethanol, and the resulting solution is sterilized by filtration to obtain a vitamin K1 solution;
0.5 g of heme is dissolved in 1 mL of 1 mol/L sodium hydroxide solution, then distilled water is added to achieve a final volume of 100 mL, the resulting solution is autoclaved at 121° C. to obtain a heme solution, and the heme solution is stored at 4° C. for use; and
5 mL of vitamin K1 solution and 10 mL of heme solution are aseptically added to the basic medium cooled to room temperature, and the pH is adjusted to 7.8 to 8.4 to obtain an enrichment medium.

4. The ELISA kit according to claim 2, wherein Clostridium novyi type B is cultivated at 37° C. for 3 days in an anaerobic environment with a gas composition of 80% nitrogen, 10% hydrogen and 10% carbon dioxide.

5. The ELISA kit according to claim 1, wherein the ELISA kit further comprises: an ELISA plate, a blocking solution, a Clostridium novyi type B-positive serum, a Clostridium novyi type B-negative serum, an enzyme-labeled secondary antibody, a chromogenic substrate solution and a stop solution.

6. The ELISA kit according to claim 5, wherein the blocking solution is 5% skimmed milk powder.

7. The ELISA kit according to claim 5, wherein the enzyme-labeled secondary antibody is HRP-labeled goat anti-rabbit antibody and HRP-labeled rabbit anti-goat antibody.

8. A method for detecting anti-Clostridium putrificum antibodies using the ELISA kit according to claim 1, wherein the method does not aim at the diagnosis and treatment of a disease, and comprises the following steps:
(1) diluting the suspension of Clostridium novyi type B 40 times with an antigen-coating buffer, and then coating the ELISA plate overnight at 4° C.;
(2) washing the ELISA plate with PBST solution, and then performing the blocking with the blocking solution at 37° C. for 2 h;
(3) adding diluted Clostridium novyi type B-positive serum, Clostridium novyi type B-negative serum and to-be-tested serum separately, and incubating the plate at 37° C. for 1 h;
(4) adding an enzyme-labeled secondary antibody diluted at 1:10,000, and incubating the plate at 37° C. for 1 h;
(5) adding a chromogenic substrate solution, and conducting reaction at 37° C. for 15 min in the dark; and
(6) adding a stop solution to stop the reaction, reading the absorbance at 450 nm, and determining the test result.

9. (canceled)

10. (canceled)

11. A method for detecting anti-Clostridium putrificum antibodies using the ELISA kit according to claim 2, wherein the method does not aim at the diagnosis and treatment of a disease, and comprises the following steps:
(1) diluting the suspension of Clostridium novyi type B 40 times with an antigen-coating buffer, and then coating the ELISA plate overnight at 4° C.;
(2) washing the ELISA plate with PBST solution, and then performing the blocking with the blocking solution at 37° C. for 2 h;
(3) adding diluted Clostridium novyi type B-positive serum, Clostridium novyi type B-negative serum and to-be-tested serum separately, and incubating the plate at 37° C. for 1 h;
(4) adding an enzyme-labeled secondary antibody diluted at 1:10,000, and incubating the plate at 37° C. for 1 h;
(5) adding a chromogenic substrate solution, and conducting reaction at 37° C. for 15 min in the dark; and
(6) adding a stop solution to stop the reaction, reading the absorbance at 450 nm, and determining the test result.

12. A method for detecting anti-Clostridium putrificum antibodies using the ELISA kit according to claim 3, wherein the method does not aim at the diagnosis and treatment of a disease, and comprises the following steps:
(1) diluting the suspension of Clostridium novyi type B 40 times with an antigen-coating buffer, and then coating the ELISA plate overnight at 4° C.;
(2) washing the ELISA plate with PBST solution, and then performing the blocking with the blocking solution at 37° C. for 2 h;
(3) adding diluted Clostridium novyi type B-positive serum, Clostridium novyi type B-negative serum and to-be-tested serum separately, and incubating the plate at 37° C. for 1 h;
(4) adding an enzyme-labeled secondary antibody diluted at 1:10,000, and incubating the plate at 37° C. for 1 h;
(5) adding a chromogenic substrate solution, and conducting reaction at 37° C. for 15 min in the dark; and
(6) adding a stop solution to stop the reaction, reading the absorbance at 450 nm, and determining the test result.

13. A method for detecting anti-Clostridium putrificum antibodies using the ELISA kit according to claim 4, wherein the method does not aim at the diagnosis and treatment of a disease, and comprises the following steps:
(1) diluting the suspension of Clostridium novyi type B 40 times with an antigen-coating buffer, and then coating the ELISA plate overnight at 4° C.;
(2) washing the ELISA plate with PBST solution, and then performing the blocking with the blocking solution at 37° C. for 2 h;
(3) adding diluted Clostridium novyi type B-positive serum, Clostridium novyi type B-negative serum and to-be-tested serum separately, and incubating the plate at 37° C. for 1 h;
(4) adding an enzyme-labeled secondary antibody diluted at 1:10,000, and incubating the plate at 37° C. for 1 h;
(5) adding a chromogenic substrate solution, and conducting reaction at 37° C. for 15 min in the dark; and
(6) adding a stop solution to stop the reaction, reading the absorbance at 450 nm, and determining the test result.

14. A method for detecting anti-Clostridium putrificum antibodies using the ELISA kit according to claim 5, wherein the method does not aim at the diagnosis and treatment of a disease, and comprises the following steps:
(1) diluting the suspension of Clostridium novyi type B 40 times with an antigen-coating buffer, and then coating the ELISA plate overnight at 4° C.;
(2) washing the ELISA plate with PBST solution, and then performing the blocking with the blocking solution at 37° C. for 2 h;
(3) adding diluted Clostridium novyi type B-positive serum, Clostridium novyi type B-negative serum and to-be-tested serum separately, and incubating the plate at 37° C. for 1 h;
(4) adding an enzyme-labeled secondary antibody diluted at 1:10,000, and incubating the plate at 37° C. for 1 h;
(5) adding a chromogenic substrate solution, and conducting reaction at 37° C. for 15 min in the dark; and
(6) adding a stop solution to stop the reaction, reading the absorbance at 450 nm, and determining the test result.

15. A method for detecting anti-Clostridium putrificum antibodies using the ELISA kit according to claim 6, wherein the method does not aim at the diagnosis and treatment of a disease, and comprises the following steps:
(1) diluting the suspension of Clostridium novyi type B 40 times with an antigen-coating buffer, and then coating the ELISA plate overnight at 4° C.;
(2) washing the ELISA plate with PBST solution, and then performing the blocking with the blocking solution at 37° C. for 2 h;
(3) adding diluted Clostridium novyi type B-positive serum, Clostridium novyi type B-negative serum and to-be-tested serum separately, and incubating the plate at 37° C. for 1 h;
(4) adding an enzyme-labeled secondary antibody diluted at 1:10,000, and incubating the plate at 37° C. for 1 h;
(5) adding a chromogenic substrate solution, and conducting reaction at 37° C. for 15 min in the dark; and
(6) adding a stop solution to stop the reaction, reading the absorbance at 450 nm, and determining the test result.

16. A method for detecting anti-Clostridium putrificum antibodies using the ELISA kit according to claim 7, wherein the method does not aim at the diagnosis and treatment of a disease, and comprises the following steps:
(1) diluting the suspension of Clostridium novyi type B 40 times with an antigen-coating buffer, and then coating the ELISA plate overnight at 4° C.;
(2) washing the ELISA plate with PBST solution, and then performing the blocking with the blocking solution at 37° C. for 2 h;
(3) adding diluted Clostridium novyi type B-positive serum, Clostridium novyi type B-negative serum and to-be-tested serum separately, and incubating the plate at 37° C. for 1 h;
(4) adding an enzyme-labeled secondary antibody diluted at 1:10,000, and incubating the plate at 37° C. for 1 h;
(5) adding a chromogenic substrate solution, and conducting reaction at 37° C. for 15 min in the dark; and
(6) adding a stop solution to stop the reaction, reading the absorbance at 450 nm, and determining the test result.

17. The method according to claim 8, wherein, in step (3), Clostridium novyi type B-positive serum, Clostridium novyi type B-negative serum and to-be-tested serum are all diluted at 1:100.

18. The method according to claim 11, wherein, in step (3), Clostridium novyi type B-positive serum, Clostridium novyi type B-negative serum and to-be-tested serum are all diluted at 1:100.

19. The method according to claim 12, wherein, in step (3), Clostridium novyi type B-positive serum, Clostridium novyi type B-negative serum and to-be-tested serum are all diluted at 1:100.

20. The method according to claim 13, wherein, in step (3), Clostridium novyi type B-positive serum, Clostridium novyi type B-negative serum and to-be-tested serum are all diluted at 1:100.

21. The method according to claim 14, wherein, in step (3), Clostridium novyi type B-positive serum, Clostridium novyi type B-negative serum and to-be-tested serum are all diluted at 1:100.

22. The method according to claim 8, wherein, in step (6), the test result is determined according to the following criterion: if OD value of the to-be-tested sample is greater than 0.531, it is determined as positive; and if OD value of the to-be-tested sample is less than 0.531, it is determined as negative.
